SEQUENCE LISTING (6 sequences)

SEQ ID NO: 1 - DNA, 1935 bp, Artificial Sequence
  Plasmid for E. coli
catatgatggcgagcgcgccgattggcagcgcgattccgcgcaacaactgggcggtgacctgcgatagcgcgcagagcggcaacgaatgcaacaaagcgattgatggcaacaaagataccttttggcataccttttatggcgcgaacggcgatccgaaaccgccgcatacctataccattgatatgaaaaccacccagaacgtgaacggcctgagcgtgctgccgcgccaggatggcaaccagaacggctggattggccgccatgaagtgtatctgagcagcgatggcaccaactggggcagcccggtggcgagcggcagctggtttgcggatagcaccaccaaatatagcaactttgaaacccgcccggcgcgctatgtgcgcctggtggcgattaccgaagcgaacggccagccgtggaccagcattgcggaaattaacgtgtttcaggcgagcagctataccgcgccgcagccgggcctgggccgctggggcccgaccattgatctgccgattgtgccggcggcggcggcgattgaaccgaccagcggccgcgtgctgatgtggagcagctatcgcaacgatgcgtttgaaggcagcccgggcggcattaccctgaccagcagctgggatccgagcaccggcattgtgagcgatcgcaccgtgaccgtgaccaaacatgatatgttttgcccgggcattagcatggatggcaacggccagattgtggtgaccggcggcaacgatgcgaaaaaaaccagcctgtatgatagcagcagcgatagctggattccgggcccggatatgcaggtggcgcgcggctatcagagcagcgcgaccatgagcgatggccgcgtgtttaccattggcggcagctggagcggcggcgtgtttgaaaaaaacggcgaagtgtatagcccgagcagcaaaacctggaccagcctgccgaacgcgaaagtgaacccgatgctgaccgcggataaacagggcctgtatcgcagcgataaccatgcgtggctgtttggctggaaaaaaggcagcgtgtttcaggcgggcccgagcaccgcgatgaactggtattataccagcggcagcggcgatgtgaaaagcgcgggcaaacgccagagcaaccgcggcgtggcgccggatgcgatgtgcggcaacgcggtgatgtatgatgcggtgaaaggcaaaattctgacctttggcggcagcccggattatcaggatagcgatgcgaccaccaacgcgcatattattaccctgggcgaaccgggcaccagcccgaacaccgtgtttgcgagcaacggcctgtattttgcgcgcacctttcataccagcgtggtgctgccggatggcagcacctttattaccggcggccagcgccgcggcattccgtttgaagatagcaccccggtgtttaccccggaaatttatgtgccggaacaggataccttttataaacagaacccgaacagcattgtgcgcgcgtatcatagcattagcctgctgctgccggatggccgcgtgtttaacggcggcggcggcctgtgcggcgattgcaccaccaaccattttgatgcgcagatttttaccccgaactatctgtatgatagcaacggcaacctggcgacccgcccgaaaattacccgcaccagcacccagagcgtgaaagtgggcggccgcattaccattagcaccgatagcagcattagcaaagcgagcctgattcgctatggcaccgcgacccataccgtgaacaccgatcagcgccgcattccgctgaccctgaccaacaacggcggcaacagctatagctttcaggtgccgagcgatagcggcgtggcgctgccgggctattggatgctgtttgtgatgaacagcgcgggcgtgccgagcgtggcgagcaccattcgcgtgacccagtaactcgag

SEQ ID NO: 2 - DNA, 1881 bp, Artificial sequence
  pyranose oxidase from Phanerochaete chrysosporium
catatgatgttcctagacacaacaccattcagagcagacgaaccatacgacgtattcatagcaggaagcggaccaataggagcaacattcgcaaaactatgcgtagacgcaaacctaagagtatgcatggtagaaataggagcagcagacagcttcacaagcaaaccaatgaaaggagacccaaacgcaccaagaagcgtacaattcggaccaggacaagtaccaataccaggataccacaaaaaaaacgaaatagaataccaaaaagacatagacagattcgtaaacgtaataaaaggagcactaagcacatgcagcataccaacaagcaacaaccacatagcaacactagacccaagcgtagtaagcaacagcctagacaaaccattcataagcctaggaaaaaacccagcacaaaacccattcgtaaacctaggagcagaagcagtaacaagaggagtaggaggaatgagcacacactggacatgcgcaacaccagaattcttcgcaccagcagacttcaacgcaccacacagagaaagaccaaaactaagcacagacgcagcagaagacgcaagaatatggaaagacctatacgcacaagcaaaagaaataataggaacaagcacaacagaattcgaccacagcataagacacaacctagtactaagaaaatacaacgacatattccaaaaagaaaacgtaataagagaattcagcccactaccactagcatgccacagactaacagacccagactacgtagaatggcacgcaacagacagaatactagaagaactattcacagacccagtaaaaagaggaagattcacactactaacaaaccacagatgcacaaaactagtattcaaacactacagaccaggagaagaaaacgaagtagactacgcactagtagaagacctactaccacacatgcaaaacccaggaaacccagcaagcgtaaaaaaaatatacgcaagaagctacgtagtagcatgcggagcagtagcaacagcacaagtactagcaaacagccacataccaccagacgacgtagtaataccattcccaggaggagaaaaaggaagcggaggaggagaaagagacgcaacaataccaacaccactaatgccaatgctaggaaaatacataacagaacaaccaatgacattctgccaagtagtactagacagcagcctaatggaagtagtaagaaacccaccatggccaggactagactggtggaaagaaaaagtagcaagacacgtagaagcattcccaaacgacccaataccaataccattcagagacccagaaccacaagtaacaataaaattcacagaagaacacccatggcacgtacaaatacacagagacgcattcagctacggagcagtagcagaaaacatggacacaagagtaatagtagactacagattcttcggatacacagaaccacaagaagcaaacgaactagtattccaacaacactacagagacgcatacgacatgccacaaccaacattcaaattcacaatgagccaagacgacagagcaagagcaagaagaatgatggacgacatgtgcaacatagcactaaaaataggaggatacctaccaggaagcgaaccacaattcatgacaccaggactagcactacacctagcaggaacaacaagatgcggactagacacacaaaaaacagtaggaaacacacactgcaaagtacacaacttcaacaacctatacgtaggaggaaacggagtaatagaaacaggattcgcagcaaacccaacactaacaagcatatgctacgcaataagagcaagcaacgacataatagcaaaattcggaagacacagaggataataactcgag

SEQ ID NO: 3 - DNA, 1845 bp, Artificial sequence
  Hexose oxidase from Chondrus crispus
catatgatgacattcagcataaaaacaagaaaaaacacagtagacaaactaaaaaacgaagaactagacctactagtaataggaggaggaataacaggagcaggagtagcactacaagcaacagcaagcggactaaaaacaggactagtagaaatgcaagacttcgcagaaggaacaagcagcagaagcacaaaactagtacacggaggaataagatacctaaaaacattcgacgtaggagtagtagcagacacagtacaagaaagagcagtagtacaaggaatagcaccacacataccaagaccattcccaatgctactaccaatatacgacgacccaagcagcacattcgacatgttcagcgtaaaaatagcaatggacctatacgacagactagcaggagtaacaggaacacaatacgcaaactacacaataagcagagaagaagtactacaaagagaaccaaacctaaaaagcgacaaactactaggagcaggagtatacctagactacgtaaacaacgacgcaagactagtaatagaaaacataaaagaagcagacgaactaggaggactaatggcaagccacgtaaaagtagtaggagtactacacgacgacaacggaagaataaacggagtaaaagcaaaagacctactaacagacgaagaattcgaaataaaagcaaaaatggtaataaacacaacaggaccatgggtaacaaaaacactaggactagacgacaaaaacggagaaggagaagtaataagaccaacaaaaggagtacacctagtagtagacagcagcagactaagagtaccacaaccaacatacttcgacagcggaataggagacggaagaatgatattcgtagtaccaagagaaaacaaaacatacttcggaacaacagacacagactacacaggagactacaaacacccaagagtagaacaagcagacgtagactacctactaaaagcaataaacaacagatacccagaagcagaaataacaataaacgacatagaagcaagctgggcaggactaagaccactactaagcggaaacggaagcagcgactacaacggaggaggaagcaacacaggaaaagtaagcgacgaaagcttcgaaaacctaataaaaacagcagtagcatacagcaaagacgaagcaagcagaggagaagtagaaaaaagcataagcagcctaaaaacagcaagcgcagaaaaaacactaagcccaagccaagtaagcagaggaagcagcctaaaagtaggagacgacggactaataacactaagcggaggaaaaataacagactacagaaaaatggcagcaggagcaatggaactaataagaaaactattcaaagaactattcaacaaagacgtaagagaagtagacagcaaaaaactacaagtaagcggaggacacttcgacccaacaaacgtagacgaaacaatgaaattctaccaaaaaatagcaatggacaaaggaatgccagcagcagacgcagcagacctagcaaacagattcggaagcaacacaagcagagtaataagctacgcagacgacgtagaagcagcaccaggactaacactagcagacacactaagcctacactacagcgtaaacgaagaaatgacactaaacctagtagacttcctactaagaagaacaaactacgtactattccacaaagacgaactaatgatgaaaaaagacgcattcgtagacgaaatggcaagaataatggaatggaacgaagaagaaaaagcagcagcagcaaaacaactagacgaaacaatagcagaaagcagcctagcatacctaaaataataactcgag

SEQ ID NO: 4 - DNA, 1935 bp, Artificial sequence; identical to SEQ ID NO: 1
  pASK-IBA35+-AlaDH for E. coli

SEQ ID NO: 5 - DNA, 4534 bp, Artificial sequence
  pASK-IBA35+-Vflu
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatggctagcagaggatcgcatcaccatcaccatcacggcgccaacaaaccgcagagctgggaagcgcgtgcggaaacctatagcctgtatggctttaccgatatgccgagcctgcatcagcgtggcaccgttgttgttacccatggcgaaggcccgtatatcgttgatgttaacggccgtcgttatctggatgcgaatagcggcctgtggaatatggttgcgggctttgatcataaaggcctgatcgatgcggcgaaagcgcagtatgaacgttttccgggctatcatgcgttttttggccgtatgagcgatcagaccgtgatgctgagcgaaaaactggttgaagtgagcccgtttgatagcggccgtgtgttttataccaacagcggcagcgaagcgaatgataccatggttaaaatgctgtggtttctgcatgcggcggaaggtaaaccgcagaaacgtaaaattctgacccgctggaacgcgtatcatggcgttaccgcggttagcgcgagcatgaccggcaaaccgtataacagcgtgtttggtctgccgctgccgggttttgttcatctgacctgcccgcattattggcgttatggcgaagaaggcgaaaccgaagaacagtttgttgcgcgtctggcgcgtgaactggaagaaaccattcagcgcgaaggcgcggataccattgcgggtttttttgcggaaccggttatgggtgcgggtggtgttattccgccggcgaaaggttattttcaggcgattctgccgatcctgcgcaaatatgatattccggtgatcagcgatgaagtgatttgcggctttggtcgtaccggtaatacctggggttgcgtgacctatgattttaccccggatgcgatcattagcagcaaaaacctgaccgcgggcttttttccgatgggtgcggttattctgggtccggaactgagcaaacgtctggaaaccgcgattgaagcgatcgaagaatttccgcatggttttaccgcgagcggtcatccggttggttgtgcgattgcgctgaaagcgatcgatgtggttatgaacgaaggcctggcggaaaatgttcgtcgtctggcgccgcgttttgaagaacgcctgaaacatattgcggaacgtccgaacattggcgaatatcgcggcattggttttatgtgggcgctggaagcggttaaagataaagcgagcaaaaccccgtttgatggcaatctgagcgtgagcgaacgtattgcgaatacctgcaccgatctgggtctgatttgtcgtccgctgggtcagagcgttgttctgtgcccgccgtttattctgaccgaagcgcagatggatgaaatgttcgataaactggaaaaagcgctggataaagttttcgcggaagttgcgtaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 6 - DNA, 4533 bp, Artificial sequence
  pASK-IBA5+-pCR6
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatggctagctggagccacccgcagttcgaaaaaggcgccaaccaaccgcaaagctgggaagcccgggccgagacctattcgctctacggtttcaccgacatgccctcggtccatcagcggggcacggtcgtcgtgacccatggcgaggggccctatatcgtcgatgtccatggccgccgctatctggatgccaattcgggcctgtggaacatggtcgcgggcttcgaccacaagggcctgatcgaggccgccaaggcgcaatacgaccgctttcccggctatcacgcctttttcggccgcatgtccgaccagaccgtgatgctgtcggaaaagctggtcgaggtctcgccattcgacaacggccgggtcttctataccaattccggctccgaggcgaacgacaccatggtcaagatgctgtggttcctgcatgccgccgagggcaagccgcaaaagcgcaagatcctgacgcgctggaacgcctatcacggcgtgaccgcggtttcggcctcgatgaccggcaagccctacaactcggtcttcggcctgccgctgcccggcttcatccacctgacctgcccgcattactggcgctatggcgaggaaggcgagaccgaggcgcaattcgtcgcccgcctggcacgcgagcttgaggataccatcacccgcgagggcgccgacaccatcgccggcttcttcgccgagccggtgatgggcgcggggggggtgatcccgccggcgaagggttatttccaggccatcctgccgatcttgcgcaagtatgacatcccgatgatctcggacgaggtgatctgcggcttcgggcgcaccggcaacacctggggctgcctgacctacgacttcatgcccgatgcgatcatctcgtccaagaacctgactgcgggcttcttcccgatgggcgccgtcatcctcgggcccgacctcgccaagcgggtcgaggccgcggtcgaggcgatcgaggagttcccgcacggcttcaccgcctcgggccatccggtcggctgcgccatcgcgctgaaggccatcgacgtggtgatgaacgaggggctggccgagaatgtccgccgcctcgcaccccgcttcgaggcggggctgaagcgcatcgccgaccgcccgaacatcggcgaataccgcggcatcggcttcatgtgggcgctggaggcggtcaaggacaagccgaccaagacccccttcgacgccaatctttcggtcagcgagcgcatcgccaatacctgcaccgatctggggctgatctgccggccgctgggccagtccatcgtgctgtgcccgcccttcatcctgaccgaggcgcagatggacgagatgttcgaaaagctggaaaaggcgctcgacaaggtctttgccgaggtggcctaaaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

(19) United States
(12) Patent Application Publication
(10) Pub. No.: US 2016/0244790 A1
(43) Pub. Date: Aug. 25, 2016

(54) ENZYMATIC AMINATION

(71) Applicants: Thomas HAAS, Muenster (DE); Jan Christoph Pfeffer, Hanau (DE); Kurt Faber, Graz (AT); Michael Fuchs, Graz (AT)

(72) Inventors: Thomas HAAS, Muenster (DE); Jan Christoph Pfeffer, Hanau (DE); Kurt Faber, Graz (AT); Michael Fuchs, Graz (AT)

(73) Assignee: Evonik Degussa GmbH, Essen (DE)

(21) Appl. No.: 15/073,105

(22) Filed: Mar. 17, 2016

Related U.S. Application Data
(62) Division of application No. 14/126,607, filed on Dec. 16, 2013, now abandoned.

(30) Foreign Application Priority Data
Jun. 15, 2011 (EP) 11170011.8

(51) Int. Cl. C12P 13/00; C12P 17/04; C12P 7/22; C12P 17/12

(52) U.S. Cl. CPC C12P 13/001; C12P 17/12; C12P 17/04; C12P 7/22

(57) ABSTRACT

A method comprising the steps (a) contacting a hydrocarbon comprising a hydroxyl group with a biological agent having oxygen-dependent and cofactor-dependent carbohydrate oxidase activity in the presence of oxygen and carbohydrate oxidase cofactor, and (b) contacting the hydrocarbon produced in step a) with a biological agent having transaminase activity and a biological agent having cofactor-dependent amino acid dehydrogenase activity in the presence of amino acid dehydrogenase cofactor and the substrate amino acid of the amino acid dehydrogenase.

The present invention relates to a method comprising the steps a) contacting a hydrocarbon comprising a hydroxy group with a biological agent having oxygen-dependent and cofactor-dependent carbohydrate oxidase activity in the presence of oxygen and carbohydrate oxidase cofactor, and b) contacting the hydrocarbon produced in step a) with a biological agent having transaminase activity and a biological agent having cofactor-dependent amino acid dehydrogenase activity in the presence of amino acid dehydrogenase cofactor and the substrate amino acid of the amino acid dehydrogenase, an aqueous mixture comprising a biological agent having oxygen-dependent and cofactor-dependent carbohydrate oxidase activity, a biological agent having transaminase activity, a biological agent having amino acid dehydrogenase activity, oxygen, the substrate amino acid of the amino acid dehydrogenase, carbohydrate oxidase cofactor and amino acid dehydrogenase cofactor and preferably a biological agent having formate or glucose dehydrogenase activity for transaminating a hydrocarbon comprising a hydroxy group, and a use of such an aqueous reaction mixture.

The era of non-renewable fossil fuels is coming to an end. While chemists have been able to rely on a virtually unlimited supply of coal, petroleum and natural gas, the availability of such resources, formed by bacteria, plancton, plant and animal matter buried in ocean sediments over millions of years, is expected to be limiting in the very near future. Moreover, burning fossil fuels has been linked with the increase in atmospheric concentrations of CO₂ and associated climate changes, most notably global warming. Therefore, next generation processes for the production of chemicals conventionally derived from fossil resources will have to start with renewable resources, i.e. materials that are easily and, in terms of geological time scales, rapidly replenishable.

Industrial biotechnology, i.e. the application of biocatalysts such as enzymes or organisms as industrial catalysts, offers alternatives to many conventional processes using fossil resources as input. Not only are biocatalysts able to convert compounds made from renewable materials, often agricultural or process wastes that would otherwise have to be disposed of, but they do not require the use of toxic compounds and, last but not least, reduce greenhouse gas emissions compared to conventional approaches.

Amines, organic derivates of ammonia, represent of a class of industrially sought-after compounds, including bulk chemicals such as aniline, often prepared by conversion of compounds made from fossil carbon sources such as halogenated alkanes or alkenes. The reactions used for the synthesis of amines depend on the use of extreme temperatures, toxic compounds such as HCN and expensive or environmentally hazardous catalysts. Examples of industrially relevant amines include aniline, a precursor to various industrial chemicals, lysine, an essential amino acid, and di-(aminomethyl)-furan (DAMF), a monomer used for the production of polymers.

The prior art teaches a chemical route towards the synthesis of DAMF and chemically related amines, involving the stepwise oxidation of HMF to DFF, followed by transformation to the corresponding dioxime and subsequent nickel-catalyzed reduction of the latter (El Hajj et al., 1987). A biotechnological route based on renewable materials such as sugars would be desirable.

Therefore, the problem underlying the present invention is to provide a system that may be used to produce amines starting with compounds comprising a hydroxyl group, preferably attached to an aromatic or aliphatic carbon ring, which compounds may be derived from renewable starting materials rather than compounds obtained by way of cracking fossil resources. Another problem underlying the present invention is to provide an entirely enzymatic route towards the production of organic amines, preferably one with a broad substrate specificity and/or improved yield and/or product specificity compared to reactions described in the prior art and one that does not require the addition of hazardous reagents and/or harsh reaction conditions.

Another problem involves the provision of a suite of reactions for the production of amines that has, in toto, a redox balance of 0.

Another problem underlying the present invention is to provide a system that may be used to produce amines starting with compounds comprising a hydroxyl group, preferably attached to an aromatic or aliphatic carbon ring, without the need to subject intermediates to purification and/or extraction procedures or have a change of solvent.

The problem underlying the present invention is solved by the subject-matter of the attached independent and dependent claims.

In a first aspect, the problem underlying the present invention is solved by a method comprising the steps
(a) contacting a hydrocarbon comprising a hydroxyl group with
a biological agent having oxygen-dependent and cofactor-dependent carbohydrate oxidase activity
in the presence of oxygen and carbohydrate oxidase cofactor, and
(b) contacting the hydrocarbon produced in step a) with
a biological agent having transaminase activity and
a biological agent having cofactor-dependent amino acid dehydrogenase activity
in the presence of amino acid dehydrogenase cofactor and the substrate amino acid of the amino acid dehydrogenase.

In an embodiment of the first aspect, the hydrocarbon comprising a hydroxyl group comprises a 5 or 6 membered ring carrying at least one substituent selected from the group comprising —(CH₂)ₓ—OH, wherein x is 0 to 4.

In an embodiment of the first aspect, the hydrocarbon comprising a hydroxyl group is selected from the group of compounds represented by formulae (I) or (II)

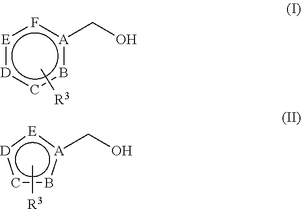

wherein up to two out of A, B, C, D, E and F are atoms each and independently selected from the group comprising N, S and O and the others are C,
wherein R³ is selected from the group comprising H, halogen, substituted or unsubstituted alkyl, alkenyl, alkynyl, —(CH₂)ₓ—CR⁴O, —(CH₂)ₓ—NH₂, —(CH₂)ₓ—NO₂, —(CH₂)ₓ—O—R⁴, —(CH₂)ₓ—CH₂SH, —(CH₂)ₓ—COOR⁴,
wherein x is 0 to 20, preferably 0 to 4, more preferably 1 to 2,
and wherein R⁴ is selected from the group comprising H and substituted or unsubstituted alkyl, cycloalkyl, aryl and heteroaryl.

In an embodiment of the first aspect, up to two out of A, B, C, D, E and F are atoms each and independently selected from the group comprising N and O and the others are C,
wherein R³ is selected from the group comprising —(CH₂)ₓ—CR⁴O, —(CH₂)ₓ—NH₂, —(CH₂)ₓ—O—R⁴ and —(CH₂)ₓ—COOR⁴,
wherein x is 0 to 4, more preferably 1 to 2,
and wherein R⁴ is selected from the group comprising H and alkyl comprising 1 to 4 carbon atoms.

In an embodiment of the first aspect, A, B, C, D, E and F are all C atoms.

In an embodiment of the first aspect, the hydrocarbon comprising a hydroxyl group is a cycloalkanol, preferably cyclohexanol.

In an embodiment of the first aspect, the oxygen-dependent and cofactor-dependent carbohydrate oxidase activity is an activity provided by an enzyme selected from the group comprising the M1 variant of galactose oxidase from Fusarium NRRL 2903, pyranose oxidase from Phanerochaete chrysosporium, hexose oxidase from Chondrus crispus and homologues thereof.

In an embodiment of the first aspect, the amino acid dehydrogenase is alanine dehydrogenase.

In an embodiment of the first aspect, the transaminase is selected from the group comprising the ortransaminases from Vibrio fluvialis and Paracoccus denitrificans and homologues thereof.

In an embodiment of the first aspect, step b) is carried out in the presence of a biological agent having formate dehydrogenase activity.

In an embodiment of the first aspect, step b) is carried out in the presence of a biological agent having glucose dehydrogenase activity.

In an embodiment of the first aspect, the redox factor produced by the biological agent having amino acid dehydrogenase activity is consumed by the biological agent having formate or glucose dehydrogenase activity.

In an embodiment of the first aspect, step a) is carried out in the presence of an H₂O₂-degrading activity.

In an embodiment of the first aspect, the H₂O₂-degrading activity is provided by a biological agent selected from the group comprising catalase and horse radish peroxidase/ABTS and homologues thereof.

In an embodiment of the first aspect, steps a) and b) are carried out simultaneously in the same reaction mixture.

In an embodiment of the first aspect, at least one component essential for the transaminase activity, preferably the biological agent having transaminase activity, is added to the reaction mixture following addition of the components essential for oxygen-dependent and cofactor-dependent carbohydrate oxidase activity.

In an embodiment of the first aspect, the level of oxygen pressure is 2 to 7 bar, preferably 3 to 5 bar.

In an embodiment of the first aspect, one or more, preferably all, of the biological agents selected from the group comprising the biological agent having oxygen-dependent and cofactor-dependent carbohydrate oxidase activity, the biological agent having transaminase activity, the biological agent having formate or glucose dehydrogenase activity and the biological agent having cofactor-dependent alanine dehydrogenase activity are associated with a viable cell.

In a second aspect, the problem underlying the present invention is solved by a use of a mixture comprising a biological agent having oxygen-dependent and cofactor-dependent carbohydrate oxidase activity, a biological agent having transaminase activity, a biological agent having amino acid dehydrogenase activity, oxygen, the substrate amino acid of the amino acid dehydrogenase, carbohydrate oxidase cofactor and amino acid dehydrogenase cofactor and preferably a biological agent having formate or glucose dehydrogenase activity for transaminating a hydrocarbon comprising a hydroxyl group, wherein one or more than one, preferably all biological agents are or comprise a heterologous polypeptide having the activity of interest.

In a third aspect, the problem underlying the present invention is solved by an aqueous reaction mixture comprising a hydrocarbon comprising a hydroxyl group, a biological agent having oxygen-dependent and cofactor-dependent carbohydrate oxidase activity, oxygen, carbohydrate oxidase cofactor, a biological agent having transaminase activity, a biological agent having amino acid dehydrogenase, amino acid dehydrogenase cofactor, the substrate amino acid of the amino acid dehydrogenase and preferably a biological agent having formate or glucose dehydrogenase activity, wherein one or more than one, preferably all biological agents are or comprise a heterologous polypeptide having the activity of interest.

In a preferred embodiment of the third aspect, each of the biological agents specified is a whole cell biocatalyst comprising one or more heterologous active polypeptides having the activities of interest, preferably one whole cell biocatalyst comprising more than one heterologous active polypeptides has all the activities of interest.

Further embodiments of the second and third aspect of the present invention comprise the embodiments of the first aspect of the present invention.

The present invention is based on the surprising finding that a sequence of reactions exists that may be used to catalyse the oxidation and subsequent transamination of a hydrocarbon comprising a hydroxyl group, wherein said hydroxyl group is converted to an amine group, based entirely on biotechnological catalysts. Moreover, the present inventors have surprisingly found that all of these reactions are fully compatible with each other and may be carried out simultaneously in the same reaction vessel, using a uniform buffer system. Moreover, the present inventors have surprisingly found a suitable sequence of reactions that may be carried out without an in toto addition or removal of electrons.

The inventive process may be used to convert a broad range of compounds. In a preferred embodiment, the term “hydrocarbon comprising a hydroxyl group”, as used herein, refers to any organic compound comprising two carbon atoms linked via a C—C bond and comprising at least one hydroxyl group, the latter preferably attached to a carbon atom. In a preferred embodiment, the carbon chain of such a hydrocarbon is selected from the group comprising cyclic and linear alkanes, alkenes, alkynes, alkyl or alkenyl aryls and alkyl or alkenyl heteroaryls.

In a preferred embodiment, the hydrocarbon comprising a hydroxyl group is selected from the group of compounds represented by formula (III):

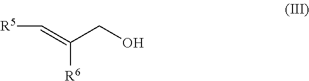

wherein R⁵ and R⁶ are each and independently any chemical group and preferably part of an aromatic ring.

In a preferred embodiment, the hydrocarbon comprising a hydroxyl group is selected from the group of compounds represented by formula (IV):

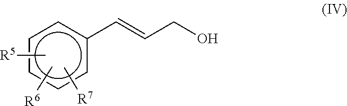

wherein each of R⁵, R⁶ and R⁷ is each and independently selected from the group comprising H, halogen, substituted or unsubstituted alkyl, alkenyl, alkynyl, —(CH₂)ₓ—CR⁴O, —(CH₂)ₓ—NH₂, —(CH₂)ₓ—NO₂, —(CH₂)ₓ—OR⁴, —(CH₂)ₓ—CH₂SH, —(CH₂)ₓ—COOR⁴,
wherein x is 0 to 20, preferably 0 to 4,
and wherein R⁴ is selected from the group comprising H and substituted or unsubstituted alkyl, cycloalkyl, aryl and heteroaryl,
and wherein up to one or two carbon atomes of the aromatic ring may each and independently be replaced by an atom selected from the group comprising N, O and S.

In a most preferred embodiment, the hydrocarbon comprising a hydroxyl group is HMF.

In a preferred embodiment, the term “biological agent”, as used herein, refers to any organism or molecule of biological origin that has the activity specified. In a more preferred embodiment, the biological agent is a purified or isolated active biological macromolecule, most preferably a peptide or polypeptide, wherein the term “isolated”, as used herein, refers to, in a particularly preferred embodiment, a biological macromolecule that has been subjected to an isolation or purification procedure and is present at a purity higher than it is in its endogenous environment such as the inside of the respective wild type cell. In another more preferred embodiment, the biological agent is a whole cell biocatalyst comprising or being associated with such a biological macromolecule. For example, the biological macromolecule may be located inside the cell or be attached to a cellular membrane, for example at the inside of the membrane in contact with the cytosol or at the outside of the membrane in contact with the environment. In a preferred embodiment, carbohydrate oxidase activity is provided by a carbohydrate oxidase or a homologue thereof. In another preferred embodiment, transaminase activity is provided by a transaminase or homologues thereof. In another preferred embodiment, catalase activity is provided by a catalase or a homologue thereof. In another preferred embodiment, amino acid, preferably alanine, dehydrogenase activity is provided by an amino acid dehydrogenase, preferably alanine dehydrogenase, or a homologue thereof. In another preferred embodiment, formate dehydrogenase activity is provided by a formate dehydrogenase or a homologue thereof. In another preferred embodiment, glucose dehydrogenase activity is provided by a glucose dehydrogenase or a homologue thereof. In another preferred embodiment, the biological agent is a polypeptide, preferably an isolated polypeptide, or a whole-cell biocatalyst. The biological agent, regardless of its nature, may be immobilised.

In a preferred embodiment, the term “presence of a cofactor”, as used herein, means that a cofactor required for an activity or enhanced activity of an activity and/or enzyme is present, either free in solution and/or tightly associated with said activity and/or enzyme.

It is clear that reaction conditions compatible with the activities involved have to be chosen, more specifically pH, buffer, buffer concentration and the like. In a preferred embodiment, the concentration of buffer, preferably sodium phosphate, is more than 25 mM, preferably 25 to 250 mM, more preferably 40 to 220 mM, most preferably 90 to 160 mM, and the pH is 5 to 9, preferably 6.5 to 8.5.

In a preferred embodiment, the term “activity”, as used herein, refers to the ability to catalyse a chemical or biological reaction. In other words, a chemical or biological reaction reaches a state of equilibrium more rapidly if a suitable activity is present. In a preferred embodiment, the activity is displayed by a catalytically active polypeptide or cell comprising such a polypeptide.

In a most preferred embodiment, the biological agent is a biological cell. The person skilled in the art is familiar with a wide range of biological cells that may be used to display a biological activity of interest. For example, the cell may be a prokaryotic cell, preferably a bacterial cell such as an Escherichia, Corynebacterium or Pseudomonas cell, most preferably an E. coli cell.

The biological cell may also be a eukaryotic cell, preferably a unicellular eukaryotic cell, most preferred a fungal biological cell, for example Candida tropicalis, Saccharomyces cerevisiae or Pichia pastoris. The person skilled in the art is also familiar with a wide range of techniques that may be used to genetically alter cells such that they express a biological or other activity of interest.

Step a) of the present invention involves the use of a biological agent having carbohydrate oxidase activity. In a preferred embodiment, the term “carbohydrate oxidase activity”, as used herein, refers to an enzymatic activity that is able to oxidise a carbohydrate, preferably a carbohydrate capable of forming, at least transiently, a five- or six-membered ring structure comprising at least one hydroxyl group, wherein at least one hydroxyl group is oxidised, preferably such that it is replaced by a carbonyl group. In a preferred embodiment, the term “hydrocarbon produced in step a)”, as used herein, means that the hydrocarbon subjected to contact with a biological agent having carbohydrate oxidase activity under the conditions specified with respect to step a) have the opportunity to first interact with the agent having carbohydrate oxidase activity, subsequently the hydrocarbon modified by way of interaction with the carbohydrate oxidase activity may proceed to react with the reagents specified with respect to step b). This does not necessitate that the reagents and activities specified with respect to step b) be absent as step a) occurs.

The person skilled in the art knows how to prepare biological agents having adequate activities, in particular by introducing suitable polypeptides into cellular hosts by means of genetic engineering using standard molecular biology techniques. Moreover, the person skilled in the art knows how to employ such cells as whole-cell catalysts or use them as a starting material to prepare more or less isolated polypeptides having the activity of interest. Finally, the person skilled in the art is familiar with ways to preserve such activities or use it to catalyse chemical reactions, for example by addition of essential cofactors, buffer reagents and adjusting pH and temperature conditions in a suitable manner. Finding such conditions is a matter of routine experimentation.

In a preferred embodiment, the carbohydrate oxidase activity is an oxygen-dependent carbohydrate oxidase activity, i.e. it depends on the presence of molecular oxygen as an oxidant. The person skilled in the art is aware of methods that may be used to supply oxygen in an aqueous solution, for example by bubbling gaseous oxygen through the aqueous solution or by adding to the aqueous solution oxygen-releasing agents.

The carbohydrate oxidase activity is, aside from oxygen, also dependent on a cofactor. In a preferred embodiment of the present invention, the term “cofactor”, as used herein, refers to a redox factor that is either tightly associated with the molecule having the activity of interest or transiently interacting with such a molecule, preferably as a substrate. In a preferred embodiment, the carbohydrate oxidase uses its cofactor as an oxidant, i.e. to sink electrons released upon oxidation of the substrate's hydroxyl group. In another preferred embodiment of the present invention, the carbohydrate oxidase cofactor is copper or a flavine cofactor, more preferably FAD⁺. In another preferred embodiment, the cofactor is selected from the group comprising FAD⁺, NAD⁺ and NADP⁺.

Step b) of the present invention involves the use of a biological agent having transaminase activity. In a preferred embodiment, the term “transaminase activity”, as used herein, is an activity capable of catalysing the conversion of a carbonyl function to an amine function. In a more preferred embodiment, the term “transamination activity”, as used herein, refers to an activity that is able to catalyse the conversion of a carbohydrate having a hydroxyl group to a carbohydrate having an amine group, with the amine function replacing the hydroxyl group.

Step b) also requires participation of an amino acid dehydrogenase activity. In a preferred embodiment, the term “amino acid dehydrogenase activity”, as used herein, refers to an activity that is able to catalyse the conversion of an acid comprising an amine function, i.e. an amino acid, preferably alanine, to an acid, wherein the amino function is replaced by carbonyl function. In a most preferred embodiment, the amino acid dehydrogenase is an alanine dehydrogenase catalysing the conversion of alanine to pyruvate. In a most preferred embodiment, the amino acid dehydrogenase is dependent on a cofactor. In a preferred embodiment, the amino acid dehydrogenase cofactor is NADH.

Finally, step b) may also involve a formate or glucose dehydrogenase activity. In a preferred embodiment, the term “formate dehydrogenase activity”, as used herein, refers to an activity capable of converting formate to CO₂, wherein a formate dehydrogenase cofactor is reduced. In a preferred embodiment, the formate dehydrogenase cofactor is NAD⁺. In a preferred embodiment, the term “glucose dehydrogenase activity”, as used herein, refers to an activity capable of oxidising glucose, wherein NADH or NADPH is produced. The use of formate or glucose dehydrogenase requires the presence of formate or glucose, respectively.

In a preferred embodiment, the term “—CR⁴O”, as used herein, refers to an aldehyd substituent in case R⁴ is hydrogen or to a keto substituent if R⁴ is an organic substituent, wherein C is covalently bound to two carbon atoms.

It is a particular strength of the present invention that the sequence of reactions devised to oxidise and transaminate an alcohol may be chosen such that it has a net redox balance of 0, i.e. it does in toto not require the addition or removal of electrons and/or oxidised or reduced redox cofactors. This is accomplished by employing an amino acid dehydrogenase, preferably an alanine dehydrogenase that releases, in an oxidised form, the cofactor used by the formate dehydrogenase to oxidase formate or by the glucose dehydrogenase to oxidise glucose. However, the oxidation and amination may also be carried out in the absence of enzymes other than those from the group comprising carbohydrate oxidase, transaminase and amino acid dehydrogenase.

The present inventors have found that it is advantageous to carry out step a) in the presence of H₂O₂-degrading activity. In a preferred embodiment, the term “H₂O₂-degrading activity”, as used herein, refers to any activity that reduces the amount of H₂O₂ present in a given aqueous solution. The person skilled in the art is familiar with many catalysts, of inorganic, organic or even biological origin, that may be used for providing H₂O₂-degrading activity.

Moreover, the present inventors have surprisingly found that the entire suite of reactions presented, i.e. no less than five independent chemical reactions, are entirely compatible with respect to each other, enabling the person skilled in the art to carry out all the reactions simultaneously in the same reaction vessel without the need to add or mix components in a particular order or to purify an intermediate.

In a preferred embodiment, homologues of any amino acid or nucleic acid sequences referred to in this application explicitly, for example by name or accession number or indeed the term “homologue of”, or implicitly, for example by functional characterisation, are within scope of the present invention. In a preferred embodiment, the term “homologue”, as used herein, comprises amino acid or nucleic acid sequences, respectively, that are 60, 70, 75, 80, 85, 90, 92, 94, 95, 96, 97, 98 or 99% identical to the reference amino acid or nucleic acid sequence. In a preferred embodiment, the term “homologue”, with regard to amino acid sequence, comprises, preferably in addition to the above sequence identity, amino acid sequences that comprise one or more conservative amino acid changes with respect to the reference sequence. In a preferred embodiment, the term “homologue” of an amino acid sequence or nucleic acid sequence comprises, preferably in addition to the above sequence identity, active portions and/or fragments of the amino acid sequence or nucleic acid sequence, respectively, and fusion constructs comprising the sequences or active portions and/or fragments thereof. In a preferred embodiment, the term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity, e.g. as a carbohydrate oxidase, ω-transaminase, catalase, formate or glucose dehydrogenase or amino acid dehydrogenase. In a preferred embodiment, the term “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, more preferably 2, most preferably one order of magnitude of the values displayed by the reference molecule with respect to a specific substrate. In a preferred embodiment, the term “homologue” of a nucleic acid comprises nucleic acids the complementary strand of which hybridises, preferably under stringent conditions, to the reference nucleic acid. Stringency of hybridisation reactions is readily determinable by one of ordinary skilled in the art, and in generally is an empirical calculation dependent on probe length, washing temperature and salt concentration. In general longer probes require higher temperatures for proper annealing, while shorter probes need lower temperatures. Hybridisation generally depends on the ability of denatured DNA to reanneal to complementary strands are present in an environment below their melting temperature. The higher the degree of desired homology between the probe and hybridisable sequence, the higher the relative temperature which may be used. As a result, higher relative temperatures would tend to make the reaction conditions more stringent, while lower temperature less so. For additional details and explanation of stringency of hybridisation reactions, see Ausubel et al. (1995). In a preferred embodiment, the term “homologue” of a nucleic acid sequence, as used herein, refers to any nucleic acid sequence that encodes the same amino acid sequence as the reference nucleic acid sequence, in line with the degeneracy of the genetic code.

In a preferred embodiment of the present invention, the term “associated with a viable cell”, as used herein, means that the biological agent characterised as such is a polypeptide located in the inside of the cell or attached to the surface or to the membrane of a cell. In a more preferred embodiment, the polypeptide is fused to a membrane protein.

In a most preferred embodiment, the hydrocarbon comprising a hydroxyl group is HMF, and the carbohydrate oxidase is preferably the M1 variant of galactose oxidase from Fusarium NRRL 2903 (Escalettes & Turner, 2008) or the pyranose oxidase from Phanerochaete chrysosporiumor or the hexose oxidase from Chondrus crispus or a homologue of any of them, and the transaminase is Vibrio flurialis or Paracoccus denitrificans transaminase or a homologue thereof.

The present invention is further illustrated by the following figures and non-limiting examples from which further features, embodiments, aspects and advantages of the present invention may be taken.

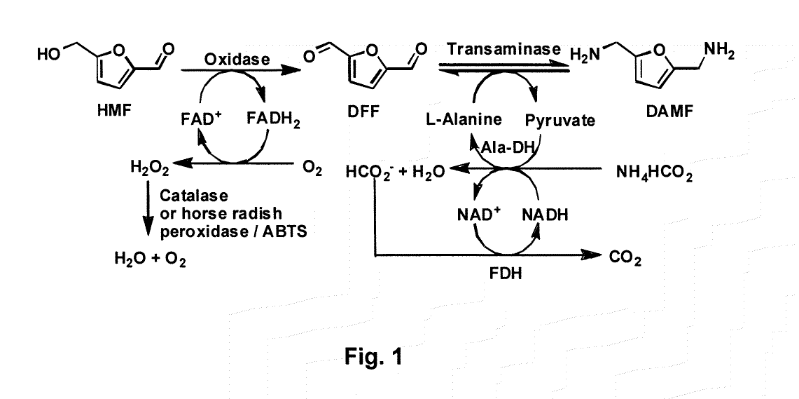

FIG. 1 depicts a scheme illustrating the inventive suite of reactions.

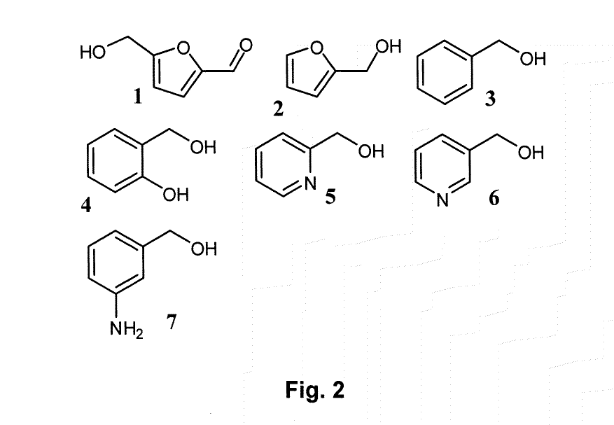

FIG. 2 depicts the range of components subjected to oxidation, as described in Example 1, and subsequent transamination, as described in Example 5, in addition to HMF.

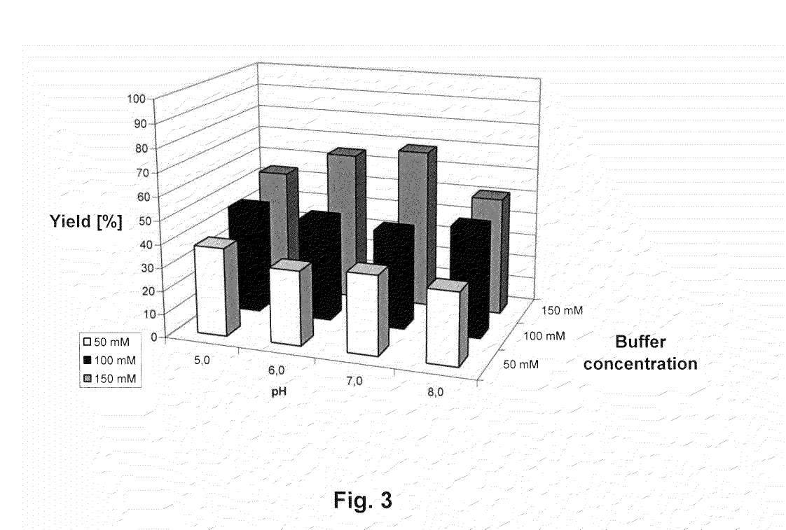

FIG. 3 shows the results of pilot experiments aiming to optimise pH and buffer concentration with respect to the oxidation reaction catalysed by the M1 variant of galactose oxidase from Fusarium NRRL2903.

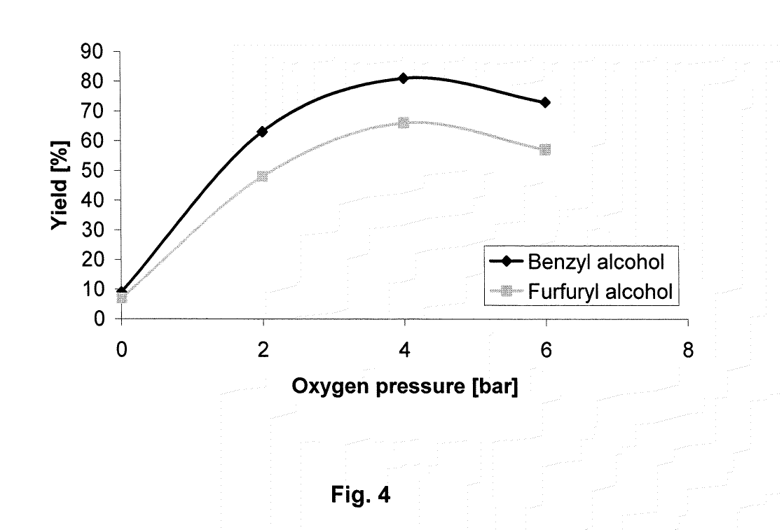

FIG. 4 shows the results of pilot experiments aiming to optimise the oxygen pressure with respect to the oxidation reaction catalysed by the M1 variant of galactose oxidase from Fusarium NRRL2903.

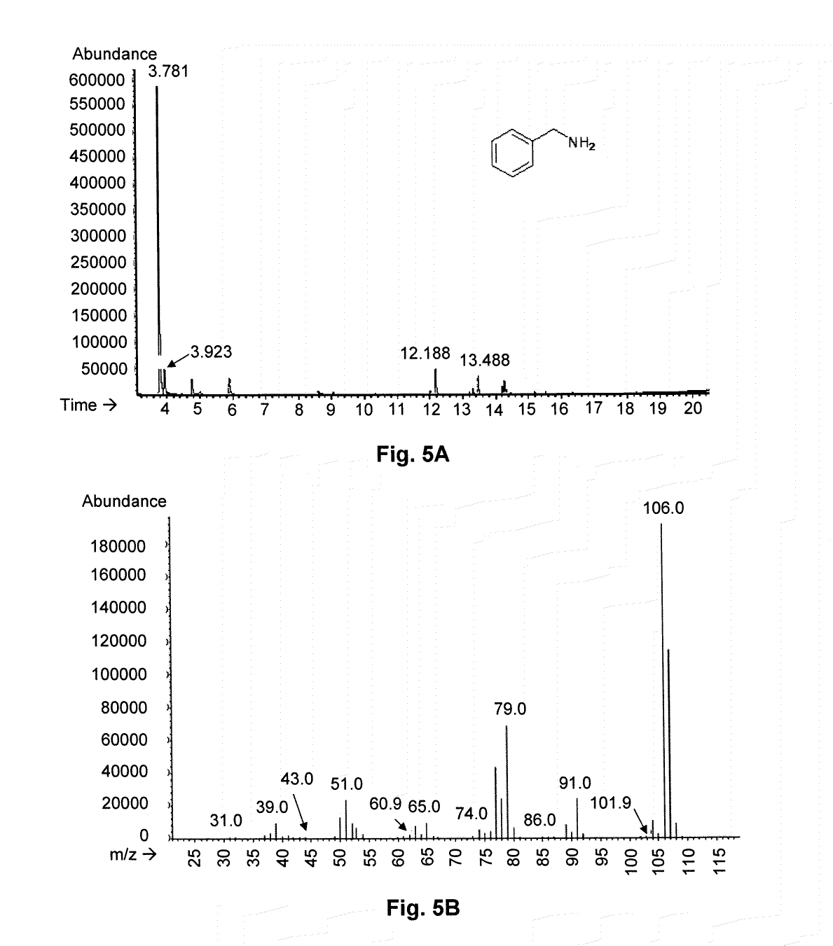

FIGS. 5A and 5B show mass spectrometry results demonstrating formation of benzylamine following oxidation and amination of benzylalcohol.

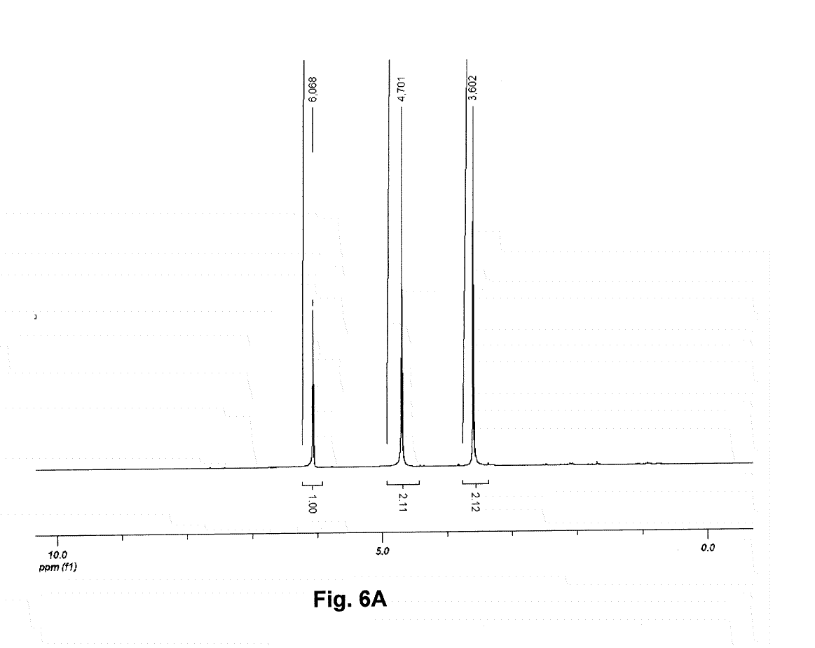

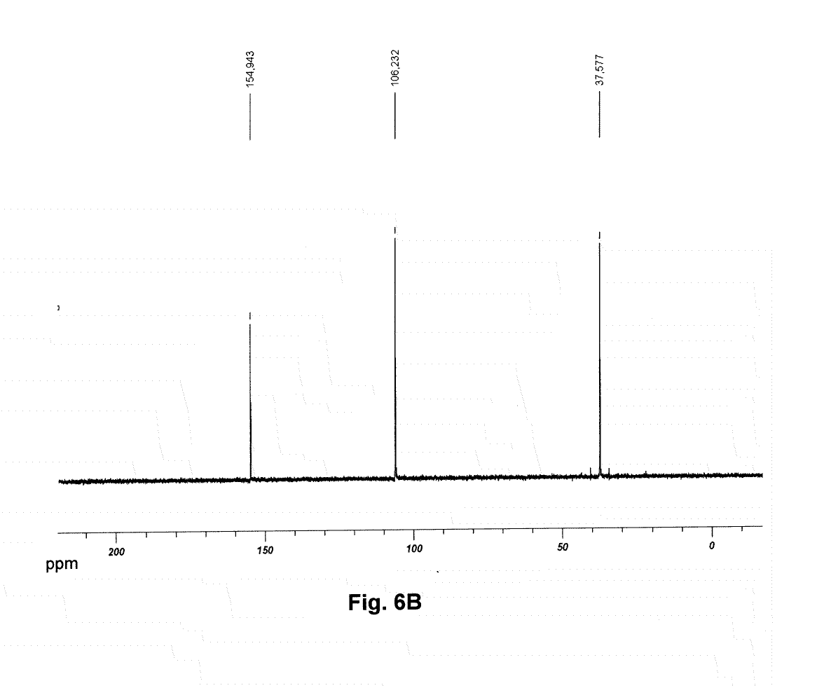

FIGS. 6A and 6B show NMR data demonstrating formation of DAMF following oxidation and amination of HMF.

### EXAMPLE 1

### Oxidation of Various Cyclic Compounds Comprising a Hydroxyl Function by Carbohydrate Oxidases

A range of cyclic compounds comprising a hydroxyl group (FIG. 2) in addition to HMF was subjected to carbohydrate oxidase oxidation as follows. A whole cell preparation of the corresponding oxidase (20 mg, lyophilised dry weight) was resuspended in the corresponding phosphate buffer [1 mL; standard conditions: galactose oxidase: 100 mM sodium phosphate, pH=7, CuSO₄.5H₂O (3 mg/mL); pyranose and hexose oxidase: 100 mM sodium phosphate, pH=8, 1 mM FAD] at 30° C. and 120 rpm for 20 min (horizontal position). Afterwards, horseradish peroxidase (Sigma, #P8125) (30 μL, 10 mg/mL), 2,2′-azino-bis(3-ethylbenzthiazoline-6-sulfonic acid) (ABTS) (30 μL, 10 mg/mL) and the respective hydrocarbon, for example HMF (5 mg, 0.04 mmol) were added and the reaction mixture was transferred to an oxygen apparatus consisting of a plexiglass cylinder (27 cm length×10 cm diameter). The apparatus was primed with oxygen (technical grade) for about 1 min and pressurized to 4 bar. The whole apparatus was shaken at room temperature, 170 rpm, 20 h (vertical position of the MG5 vial). As for catalase, 20 μL of the stock solution [Catalase from Micrococcus lysodeikticus (ca. 170000 U/mL, Sigma Aldrich #60634), Catalase from Corynebacterium glutamicum (>500000 U/mL, Sigma Aldrich #02071)] were used instead of horseradish peroxidase and 2,2′-azino-bis(3-ethylbenzthiazoline-6-sulfonic acid) (ABTS).

Various carbohydrate oxidases, more specifically galactose oxidase from Fusarium NRRL 2903 (M1 variant) (Escalettes et al., 2008), pyranose oxidase from Phanerochaete chrysosporium and hexose oxidase from Chondrus crispus, used in recombinant form in E. coli BL21(DE3) as lyophilized whole cells and obtained as described in Example 7, could be shown to convert the hydroxy groups of a range of cyclic substrates, including HMF, benzyl alcohol and ortho-hydroxybenzyl alcohol, to the corresponding carbonyl groups.

### EXAMPLE 2

### Dependency of the Oxidation Step on pH and Buffer Concentration

The reaction was carried out in aqueous solution in phosphate buffer using the M1 variant of galactose oxidase from Fusarium NRRL 2903 under standard conditions, i.e. 40 mmol HMF, 10 mg whole-cell preparation of galactose oxidase, ABTS (30 μL, 10 mg/mL), horse radish peroxidase (30 μL, 10 mg/mL) and 100 mM sodium phosphate buffer pH 7. The oxygen pressure was 4 bar. The optimum pH was 7.0 for galactose oxidase (Tab. 2, FIG. 3) As for catalase, 20 μL of the stock solution [Catalase from Micrococcus lysodeikticus (ca. 170000 U/mL, Sigma Aldrich #60634), Catalase from Corynebacterium glutamicum (>500000 U/mL, Sigma Aldrich #02071)] were used instead of horseradish peroxidase and 2,2′-azino-bis(3-ethylbenzthiazoline-6-sulfonic acid) (ABTS).

A similar suite of experiments was carried out and showed that the optimal pH value is 8.0 if pyranose oxidase is used. However, oxidation takes place over a wide pH range from 5.0 to 9.0 in the case of both enzymes.

### EXAMPLE 3

### Effect of Various Buffer Concentrations on the Oxidation Steps

A range of experiments was carried out under standard conditions as specified in Example 1, except for the fact that various concentrations of buffer were used. It could be shown that higher buffer strengths led to an increase in conversion (FIG. 3).

TABLE 2
Optimisation of pH und buffer concentration using the M1
variant of galactose oxidase from Fusarium NRRL2903.

| | | buffer strength | yield |
|---|---|---|---|
| Entry | pH | [mM] | [%] |
| 1 | 5.0 | 50 | 38 |
| 2 | 6.0 | 50 | 32 |
| 3 | 7.0 | 50 | 34 |
| 4 | 8.0 | 50 | 31 |
| 5 | 5.0 | 100 | 46 |
| 6 | 6.0 | 100 | 44 |
| 7 | 7.0 | 100 | 42 |
| 8 | 8.0 | 100 | 47 |
| 9 | 5.0 | 150 | 54 |
| 10 | 6.0 | 150 | 65 |
| 11 | 7.0 | 150 | 69 |
| 12 | 8.0 | 150 | 51 |

### EXAMPLE 4

### Effect of Elevated Levels of Oxygen on the Oxidation Step

Pilot experiments were carried out to investigate the dependency of the reaction on the oxygen pressure applied. More specifically, 40 mmol of two substrates, benzyl alcohol and furfuryl alcohol, were subjected to oxidation by the galactose oxidase as specified in Example 1, horse radish peroxidase (30 μL, 10 mg/mL) and ABTS (30 μL, 10 mg/mL) in 100 mM sodium phosphate buffer pH 7. The application of oxygen at elevated pressure was shown to be optimal at 4 bar (FIG. 4).

### EXAMPLE 5

### One-Pot Transamination of Oxidation Products of Various Cyclic Compounds Comprising a Hydroxyl Group in the Oxidation Reaction Mixture

Following incubation of the reaction mixture in Example 1 for 20 h, the transamination reaction mix consisting of transaminase, L-alanine, alanine dehydrogenase, NADH and the corresponding recycling system (see above) was added and the reaction was shaken for another 20 h. DFF was transaminated employing the recombinant transaminase from Vibrio fluvialis (Vf-ωTA) or Paracoccus denitrificans (pCR6) transaminases, see WO 2010/089171 for details of transaminases

The transaminases were used as lyophilized whole cell preparation (recombinantly in E. coli), alanine dehydrogenase from Bacillus subtilis was purified prior to use and formate dehydrogenase was obtained from Codexis (#24.11, H62411.01), see Example 7.

Reaction conditions were as follows:

Whole cell preparation of galactose oxidase from Fusarium NRRL 2903 (20 mg, lyophilised dry weight) was resuspended in sodium phosphate buffer (500 μL, 100 mM, pH=7.0, 3 mg/mL CuSO₄.5H₂O) at 30° C. and 120 rpm, 30 min. Horseradish peroxidase (15 μL, 10 mg/mL), ABTS (15 μL, 10 mg/mL) and HMF (5 mg, 0.04 mmol) were added and the reaction mixture was placed into the oxygen apparatus (vertical position, room temperature, 4 bar O₂, 170 rpm) for 20 h. The reaction mixture was transferred to Eppendorf vials. A solution of whole cell preparation of ω-transaminase from Vibrio fluvialis (20 mg, lyophilised dry weight) in Pᵢ-buffer (500 μL, 100 mM, pH=7.0, 2 mM PLP, 200 mmol L-alanine, 140 mmol ammonium formate) was added, followed by formate dehydrogenase (20 μL, Codexis) and purified alanine dehydrogenase from Bacillus subtilis (20 μL, 7.5 mg protein/mL). The reaction mixture was shaken at 30° C. and 120 rpm in a horizontal position for 20 h. The apparatus was primed with oxygen for about 1 min and pressurized to 4 bar. The reaction could be reproduced using as carbohydrate oxidase pyranose and hexose oxidase. The reaction conditions were identical except for the fact that the pH value was adjusted to 8, and 1 mM FAD was added rather than CuSO₄.5H₂O.

This example illustrates that the transamination reaction is fully compatible with the conditions of the oxidation reaction mixture. Irrespective of the transaminase used, HMF was converted to DAMF at a yield of more than 80% after 20 h reaction time, more specifically 81% and 83% if Vf-ωTA or pCR6 transaminases were used. Other compounds were oxidised and transaminated at a total combined yield as shown in Tab. 3. DFF formed during the oxidation step was quantitatively converted to DAMF. Remaining HMF did not inhibit the transamination, but was converted to the corresponding hydroxymethylamine. In an analogous fashion, HMF was converted by all transaminases to [5-(aminomethyl)furan-2-yl]methanol (AMFM). The rate of conversion and the identity of the products were determined using GC-MS.

TABLE 3
A range of substrates, see FIG. 2, were subjected to the sequential
oxidation and transamination steps as described in examples 1 and 5.

| | Rate of conversion |
|---|---|
| Substrate | [%] |
| 1 | 48 |
| 2 | 56 |
| 3 | 51 |
| 4 | Could not be extracted |
| 5 | 13 |
| 6 | 33 |
| 7 | 33 |

### EXAMPLE 6

### Simultaneous Oxidation by Carbohydrate Oxidases and Transamination of Various Cyclic Compounds Comprising a Hydroxyl Function

The same experimental setup as outlined in Example 1 was added, except for the fact that HMF was used as a substrate and all reagents required for the oxidation and transamination were added right at the beginning, including pyranose oxidase from Phanerochaete chrysosporium, horseradish peroxidase and ABTS, ω-transaminase from Vibrio fluvialis, formate dehydrogenase and alanine dehydrogenase from Bacillus subtilis. 1 mM FAD was present and the pH was 8. The reaction was allowed to proceed for 24 h.

The reaction was reproduced using as carbohydrate oxidase hexose oxidase under identical reaction conditions.

### EXAMPLE 7

### Oxidation and Transamination by Alternative Transaminases, Amino Acid Dehydrogenases in the Absence/Presence of Formate Dehydrogenase, Comprising Isolation of the Oxidation Product

The oxidation reaction was performed as described in Example 1, using compounds 1, 2 and 3, as depicted in Table 4, as substrates. However, down stream reactions, more specifically the transamination, were not carried out simultaneously in the same reaction vessel, but the product of the oxidation reaction was isolated by extraction using EtOAc (2×500 μL), the conversion determined using GC-FID and the solvent was removed via a positive stream of air. The oxidation product remaining in the vessel was subsequently used for the transamination step, which was performed as described in example 5 of the application as filed, with modifications and results as summarised in Table 5.

Five experiments were conducted with each substrate, wherein the first experiment (entries 1, 6 and 11) was each carried out under standard conditions, i.e. those described in the patent application, the second reaction was carried out in the absence of formate dehydrogenase (FDH) in the presence of equimolar NADH cofactor, the third reaction was carried out using glutamic dehydrogenase from bovine liver rather than alanine dehydrogenase from Bacillus subtilies. Alternative transaminases, namely ω-transaminase from Alcaligenes denitrificans Y2k-2 (Ad-ωTA) and ω-transaminase from Pseudomonas putida KT2440 (Gene PP5182, pCR7)] were used in experiments 4 and 5, respectively. In addition, another amino acid dehydrogenase, glutamic dehydrogenase from Candida utilis, was used in experiment 5.

The data presented illustrate that carrying out the inventive sequence of reactions in the same reaction vessel is optional. Moreover, alternative transaminases and amino acid dehydrogenases may be used. Finally, the presence of formate dehydrogenase is another optional feature.

TABLE 4
Substrates used in Example 7.

| | |
|---|---|
| 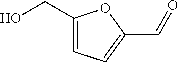 | 1 |
| 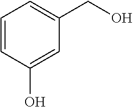 | 2 |
| 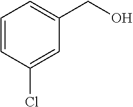 | 3 |

TABLE 5
Seqential Oxidation - Transamination sequence with isolation of the intermediate.
Entry Substrate Reaction conditions - Transamination Conv. [%]^{a}

| | | FDH^{b} | AA-DH^{c} | ωTA^{d} | NADH | Ox.^{e} | Alc. | Ald. | Amine |
|---|---|---|---|---|---|---|---|---|---|
| 1 | 1 | + | Ala-DH | Vf-ωTA | — | >99 | <1 | <1 | >99^{f} |
| 2 | 1 | − | Ala-DH | Vf-ωTA | 2 equiv. | >99 | <1 | <1 | >99^{f} |
| 3 | 1 | + | BL-Glu-DH. | Vf-ωTA | — | >99 | <1 | <1 | >99^{f} |
| 4 | 1 | + | Ala-DH | Ad-ωTA | — | >99 | <1 | <1 | >99^{f} |
| 5 | 1 | + | CU-Glu-DH. | pCR7 | — | >99 | <1 | <1 | >99^{f} |
| 6 | 2 | + | Ala-DH | Vf-ωTA | — | >99 | <1 | <1 | >99^{g} |
| 7 | 2 | − | Ala-DH | Vf-ωTA | 1 equiv. | >99 | <1 | <1 | >99^{g} |
| 8 | 2 | + | BL-Glu-DH | Vf-ωTA | — | >99 | <1 | <1 | >99^{g} |
| 9 | 2 | + | Ala-DH | Ad-ωTA | — | >99 | <1 | <1 | >99^{g} |
| 10 | 2 | + | CU-Glu-DH | pCR7 | — | >99 | <1 | <1 | >99^{g} |
| 11 | 3 | + | Ala-DH | Vf-ωTA | — | >99 | <1 | 5 | 95 |
| 12 | 3 | − | Ala-DH | Vf-ωTA | 1 equiv. | >99 | <1 | <1 | >99 |
| 13 | 3 | + | BL-Glu-DH | Vf-ωTA | — | >99 | <1 | 3 | 97 |
| 14 | 3 | + | Ala-DH | Ad-ωTA | — | >99 | <1 | 119 | 89 |
| 15 | 3 | + | CU-Glu-DH. | pCR7 | — | >99 | 15 | <1 | 85 |

^{a}Conversion was determined using GC-FID (oxidation) and GC-MS (2^{nd} step);
^{b}+ = FDH was added, − = no FDH was added;
^{c}Ala-DH = alanine dehydrogenase from Bacillus subtilis, BL-Glu-DH = glutamic dehydrogenase from Bovine Liver, CU-Glu-DH = Glutamic Dehydrogenase from Candida utilis; Vf-ωTA = ω-transaminase from Vibrio fluvialis, Ad-ωTA = ω-transaminase from Alcaligenes denitrificans Y2k-2, pCR7 = ω-transaminase from Pseudomonas putida KT2440 (Gene PP5182);
^{e}conversion of the oxidative reaction step;
^{f}conversion towards the diamine is given, determined by derivatised GC-MS sample and HPLC-UV-analysis;
^{g}conversion was determined by derivatised GC-MS-sample.

### EXAMPLE 8

### Use of Glucose Dehydrogenase In Lieu of Formate Dehydrogenase and Further Substrates for the Oxidation/transamination Reaction

A range of substrates, see Table 6 was subjected to the oxidation and transamination reaction as described in Example 6 except for the fact that glucose dehydrogenase (X-zymes, #B4A, 20 U), ammonium chloride (200 mM, 5 equiv.) and glucose (140 mM, 2.8 equiv.)] were used instead of FDH and ammonium formate.

It could be demonstrated that additional substrates, notably with halogen substituents, may be oxidised and transaminated using the inventive method.

TABLE 6
Substrates used in Example 6 and yields observed.

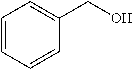

>99% conv.

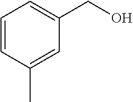

>99% conv.

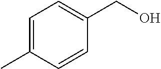

82% conv.

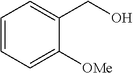

18% conv.

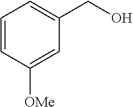

81% conv.

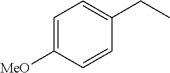

44% conv.

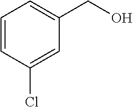

96% conv.

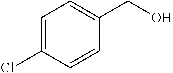

68% conv.

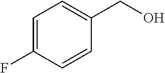

75% conv.

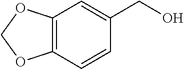 [SMILES: OCc1ccc2c(c1)OCO2]

35% conv.

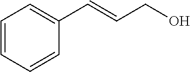

92% conv.

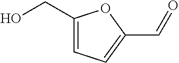

>97% conv.
D [symbol]
H [symbol] [symbol]
indicates data missing or illegible when filed

### EXAMPLE 9

### Analytical Methods

Sample Preparation for HPLC-UV Measurements without Derivatisation.

The reaction mixture was heated to 95° C. for 10 min to precipitate enzymes, diluted 1/1 with MeCN and centrifuged (13000 rpm, 3 min). Samples of 100 μL were taken, diluted with 1 mL H₂O/MeCN 1/1 containing 0.1% TFA and subjected to HPLC-UV analysis using the following setup. Column: Phenomenex LUNA 5 μm C18(2) 100 A (250×4.60 mm); oven temp.: 30° C., flow: 0.25 mL/min, eluent A: H₂O (0.1% TFA), eluent B: MeCN (0.1% TFA); Gradient: 0-4 min 30% B, 4-30 min 30-100% B, 30-36 min 100% B, 36-46 min 30% B.

Sample Derivatisation for GC-MS Measurements.

Samples of 500 μL were treated with DMAP (500 μL, 10% in MeOH). Ethyl chloroformate (200 μL) was added and the mixture was incubated at 60° C. and 300 rpm for 2 h. Extraction with dichloromethane (2×500 μL) and drying of the combined organic phase over Na₂SO₄ gave a sample which was subjected to GC-MS analysis under following conditions: Agilent 7890A GC system equipped with an Agilent 975C mass-selective detector (El 70 eV) and a HP-5-MS column (30 m×0.25 mm×0.25 μm film) using helium as carrier gas at a flow of 0.55 mL/min. The following temperature program was used: 100° C., hold for 0.5 min, 10° C./min, to 300° C.

FMOC Derivatisation for HPLC-UV Measurements.

Sample preparation was performed to a standard procedure involving formation of FMOC derivatives in a glass HPLC vial, addition of borate buffer pH 9, addition of FMOC reagent and removing the FMOC reagent excess by way of addition of EVA reagent, addition of dilution buffer. The sample was subsequently subjected to HPLC analysis using the following conditions. Column: Phenomenex LUNA 5 μm C18(2) 100 A (250×4.60 mm); oven Temp. 30° C., flow: 0.5 mL/min, eluent A: H₂O (0.1% TFA), eluent B: MeCN (0.1% TFA); Gradient: 0-2 min 30% B, 2-15 min 30-100% B, 15-25 min 100% B, 25-30 min 30% B.

### EXAMPLE 10

### Preparation of Enzymes

Genes were optimized and synthesized by GeneArt and delivered in a pMAL vector with ampicillin resistence. Protein-encoding insert was cut via Ndel and Xhol restriction enzymes (Fermentas) and ligated in a pET21a expression vector via T4 Ligase (Fermentas). If oxidases were used, enzymes were prepared using pET16b or pET21a plasmid constructs as follows: a fresh single colony from an LB agar plate supplemented with 100 mg/ml Ampicillin and used to grow E. coli strain BL21 comprising the respective plasmid was used to inoculate 50 ml of an over night culture supplemented with the same amount of Ampicillin. Following incubation of said culture over night at 120 rpm and 30° C. 4 ml of the over night culture was used to inoculate 600 ml of the same medium in a 3 I flask. The flask was shaken at 37° C. and 120 rpm until an OD550 of 0.6 was reached, i.e. approximately two to four hours. The expression of the respective enzyme was subsequently induced by addition of 0.6 ml IPTG (Isopropyl-b-D-thiogalactopyranosid, 238 mg/ml), followed by incubation over night at 20° C. and 120 rpm under vigorous shaking. Cells were then spun down at 8000 rpm and 4° C. for 20 minutes. The supernatant was disposed of, the pellet was washed using physiological NaCl solution, spun down and lyophilised.

If other enzymes were used, constructs comprising the pASK-IBA35+ (IBA Biotagnology, Germany) plasmid backbone were used. A fresh single colony from an LB agar plate supplemented with 100 mg/ml Ampicillin and used to grow E. coli strain BL21 comprising the respective plasmid was used to inoculate 50 ml of an over night culture supplemented with the same amount of Ampicillin. Following incubation of said culture over night at 180 rpm and 30° C. 4 ml of the over night culture was used to inoculate 2 I of the same medium in a 5 I flask. The flask was shaken at 37° C. and 120 rpm until an OD550 of 0.6 was reached, i.e. approximately two to four hours. The expression of the respective enzyme was subsequently induced by addition of 0.2 ml Anhydrotetracyclin (2 mg/ml), followed by incubation for three hours at 30° C. and 180 rpm under vigorous shaking. Cells were then spun down at 5000 rpm and 4° C. for 20 minutes. The supernatant was disposed of, the pellet was washed using physiological NaCl solution, spun down and lyophilised.

In the case of alanine dehydrogenase, 4 g of the lyophilised cells were resuspended in phosphate buffer (50 mM NaH₂PO₄, 300 mM NaCl, 10 mM Imidazol) and lysed using ultrasonication (1 s pulse followed by incubation for 4 s, repeated for a total of 10 minutes). The cell lysate was spun down twice (16000 rpm, 20 min, 4° C.), filtered using a syringe filter and applied to a His-trap column having a bed volume of 5 ml. Protein binding unspecifically was removed by applying phosphate buffer (50 mM NaH₂PO₄, 300 mM NaCl) comprising 20 mM imidazole. Protein was eluted using the same buffer, with a gradient from 50 mM to 80 mM imidazole being applied, followed by application of 60 ml of the same buffer comprising 150 mM imidazole Fractions comprising pure enzyme were pooled, dialyses against phosphate buffer to remove imidazole and concentrated via Vivaspin tubes and centrifugation at 4000 rpm.

### REFERENCES

T. El Hajj, A. Masroua, J. C. Martin, and G. Decotes, Bull. Soc. Chim. Fr., 1987, 855-60
F. M. Ausubel (1995), Current Protocols in Molecular Biology. John Wiley & Sons, Inc.
F. Escalettes; N. J. Turner, ChemBioChem 2008, 9, 857-860
R. Huang; W. Qi; R. Su; Z. He, Chem. Commun. 2009, 46, 1115-1117.
D. Koszelewski; I. Lavandera; D. Clay; G. M. Guebitz; D. Rozzell; W. Kroutil, Angew. Chem. Int. Ed. 2008, 47, 9337-9340.
P. D. Hanke, 2009, WO 2009/023174 A2; Chem. Abstr. 150:235749.
F. Koopman; N. Wierckx; J. H. de Winde; H. J. Ruijssenaars, Bioresource Technol. 2010, 101, 6291-6296.
M. P. J. van Deurzen; F. van Rantwijk; R. A. Sheldon, J. Carbohydr. Chem. 1997, 16, 299-309.
T. H. de Koker; M. D. Mozuch; D. Cullen; J. Gaskell; P. J. Kersten, Appl. Environ. Microbiol. 2004, 70, 5794-5800.

1. A method, comprising:
(a) contacting a hydrocarbon comprising a hydroxyl group with a first polypeptide having an oxygen-dependent and cofactor-dependent carbohydrate oxidase activity or a whole-cell biocatalyst comprising the first polypeptide in the presence of oxygen and carbohydrate oxidase cofactor; and
(b) contacting a hydrocarbon produced in the contacting (a) with a second polypeptide having a transaminase activity or a whole-cell biocatalyst comprising the second polypeptide and a third polypeptide having a cofactor-dependent amino acid dehydrogenase activity or a whole-cell biocatalyst comprising the third polypeptide in the presence of an amino acid dehydrogenase cofactor and a substrate amino acid for an amino acid dehydrogenase,
wherein the hydrocarbon comprising a hydroxyl group comprises a 5 or 6 membered ring carrying at least one substituent of —(CH₂)ₓ—OH, where x is 0 to 4, and
the first polypeptide is selected from the group consisting of an M1 variant of galactose oxidase from Fusarium NRRL 2903, pyranose oxidase from Phanerochaete chrysosporium, hexose oxidase from Chondrus crispus and a homologue thereof.

2. The method according to claim 1,
wherein the hydrocarbon comprising a hydroxyl group has formula (I) or (II): 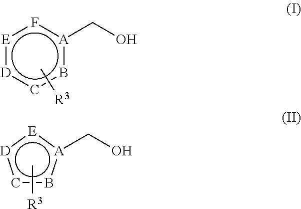
wherein one or two of A, B, C, D, E and F are atoms each and independently selected from the group consisting of N, S and O and others are C,
R³ is selected from the group consisting of H, halogen, substituted or unsubstituted alkyl, alkenyl, alkynyl, —(CH₂)ₓ—CR⁴O, —(CH₂)ₓ—NH₂, —(CH₂)ₓ—NO₂, —(CH₂)ₓ—O—R⁴, —(CH₂)ₓ—CH₂SH, and —(CH₂)ₓ—COOR⁴,
where x is 0 to 20, and
R⁴ is selected from the group consisting of H, a substituted or unsubstituted alkyl, a substituted or unsubstituted cycloalkyl, a substituted or unsubstituted aryl, and a substituted or unsubstituted heteroaryl.

3. The method according to claim 2,
wherein one or two of A, B, C, D, E and F are atoms each and independently selected from the group consisting of N and O, and others are C,
R³ is selected from the group consisting of —(CH₂)ₓ—CR⁴O, —(CH₂)ₓ—NH₂, —(CH₂)ₓ—O—R⁴, and —(CH₂)ₓ—COOR⁴,
where x is 0 to 4,
and R⁴ is selected from the group consisting of H₂ and an alkyl comprising 1 to 4 carbon atoms.

4. The method according to claim 3,
wherein the hydrocarbon comprising a hydroxyl group is a cycloalkanol.

5. The method according to claim 1,
wherein the amino acid dehydrogenase is alanine dehydrogenase.

6. The method according to claim 1,
wherein the transaminase is selected from the group consisting of ω-transaminase from Vibrio fluvialis, ω-transaminase from Paracoccus denitrificans, and a homologue thereof.

7. The method according to claim 1,
wherein the contacting (b) is carried out in the presence of a fourth polypeptide having a formate dehydrogenase activity or a whole-cell biocatalyst comprising the fourth polypeptide.

8. The method according to claim 1,
wherein the contacting (b) is carried out in the presence of a fifth polypeptide having a glucose dehydrogenase activity or a whole-cell biocatalyst comprising the fifth polypeptide.

9. (canceled)

10. The method according to claim 7,
wherein a redox factor produced by the amino acid dehydrogenase activity of the third polypeptide is consumed by the formate dehydrogenase activity of the fourth polypeptide.

11. The method according to claim 1,
wherein the contacting (a) is carried out in the presence of a compound having an H₂O₂-degrading activity.

12. The method according to claim 11,
wherein the compound is a seventh polypeptide comprising catalase and horse radish peroxidase/ABTS, and a homologue thereof or a whole-cell biocatalyst comprising the seventh polypeptide.

13. The method according to claim 1, wherein the contacting (a) and the contacting (b) are carried out simultaneously in one reaction mixture.

14. The method according to claim 12,
wherein the contacting (a) and the contacting (b) are carried out in a reaction mixture, and the second polypeptide or the whole-cell biocatalyst comprising the second polypeptide is added to the reaction mixture after the first polypeptide or the whole-cell biocatalyst comprising the first polypeptide is added to the reaction mixture.

15. The method according to claim 1,
wherein a level of oxygen pressure during the contacting (a) and the contacting (b) is from 2 to 7 bar.

16. The method according to claim 1,
wherein at least one of the first, second, and third polypeptides is associated with a viable cell.

17. A method for transaminating a hydrocarbon comprising a hydroxyl group, the method comprising:
transaminating the hydrocarbon with use-of a mixture comprising
a first polypeptide having an oxygen-dependent and cofactor-dependent carbohydrate oxidase activity or a whole-cell biocatalyst comprising the first polypeptide,
a second polypeptide having a transaminase activity or a whole-cell biocatalyst comprising the second polypeptide,
a third polypeptide having an amino acid dehydrogenase activity or a whole-cell biocatalyst comprising the third polypeptide,
oxygen,
a substrate amino acid for an amino acid dehydrogenase,
carbohydrate oxidase cofactor, and
an amino acid dehydrogenase cofactor,
wherein the hydrocarbon comprising a hydroxyl group comprises a 5 or 6 membered ring carrying at least one substituent of —(CH₂)ₓ—OH,
where x is 0 to 4, and
the first polypeptide is selected from the group consisting of an M1 variant of galactose oxidase from Fusarium NRRL 2903, pyranose oxidase from Phanerochaete chrysosporium, hexose oxidase from Chondrus crispus, and a homologue thereof.

18. An aqueous reaction mixture, comprising:
a hydrocarbon comprising a hydroxyl group;
a first polypeptide having an oxygen-dependent and cofactor-dependent carbohydrate oxidase activity or a whole-cell biocatalyst comprising the first polypeptide;
oxygen;
a carbohydrate oxidase cofactor;
a second polypeptide having a transaminase activity or a whole-cell biocatalyst comprising the second polypeptide;
a third polypeptide having an amino acid dehydrogenase activity or a whole-cell biocatalyst comprising the third polypeptide;
an amino acid dehydrogenase cofactor; and
a substrate amino acid for an amino acid dehydrogenase,
wherein at least one of the first, second, and third polypeptides and the whole-cell biocatalysts comprising the first, second and third polypeptides comprises, respectively, a heterologous polypeptide having a respectively activity,
the hydrocarbon comprising a hydroxyl group comprises a 5 or 6 membered ring having at least one substituent of —(CH₂)ₓ—OH, where x is 0 to 4, and
the first polypeptide is selected from the group consisting of an M1 variant of galactose oxidase from Fusarium NRRL 2903, pyranose oxidase from Phanerochaete chrysosporium, hexose oxidase from Chondrus crispus, and homologues a homologue thereof.
